(19) United States
(12) Patent Application Publication
(10) Pub. No.: US 2023/0414556 A1
(43) Pub. Date: Dec. 28, 2023

(54) NANO-HEPATIC TARGETED EPIGALLOCATECHIN GALLATE IN DYSLIPIDEMIA

(71) Applicant: Shaker A. Mousa, Wynantskill, NY (US)

(72) Inventor: Shaker A. Mousa, Wynantskill, NY (US)

(21) Appl. No.: 18/212,228

(22) Filed: Jun. 21, 2023

Related U.S. Application Data
(60) Provisional application No. 63/355,804, filed on Jun. 27, 2022.

(51) Int. Cl. A61K 31/352; A61K 9/51; A61K 31/05; A61K 31/12; A61K 31/375; A61P 3/06

(52) U.S. Cl. CPC A61K 31/352; A61K 9/5161; A61K 9/5123; A61K 31/05; A61K 31/12; A61K 31/375; A61P 3/06

(57) ABSTRACT

A nanoparticle structure and a method for lowering lipids in a mammal by administering the nanoparticle structure to the mammal. The nanoparticle structure includes a hepatic targeted nanoparticle and one or more substances encapsulated within the hepatic targeted nanoparticle. The nanoparticle includes chitosan and glycyrrhetinic acid (GA) covalently linked by an amide bond which occurs between an amino group of chitosan and a carboxylic acid group of GA. The one or more substances comprise (−)-Epigallocatechin-3-gallate (EGCG).

### RELATED APPLICATIONS

The present invention claims priority to U.S. Provisional No. 63/355,804, filed on Jun. 27, 2022, which is incorporated herein by reference in its entirety.

### TECHNICAL FIELD

Embodiments of the present invention pertain to a nanoparticle structure and to administering the nanoparticle structure to a mammal for lowering lipids in the mammal.

### BACKGROUND

(−)-epigallocatechin gallate (EGCG), which is a polyphenol, has been associated treatment of hyperlipidemia and reducing the risk of cardiovascular diseases. A lipid lowering effect of EGCG is mediated by inhibition of intestinal lipid absorption and up regulation hepatocytes low-density lipoprotein (LDL) receptors. The stability of EGCG is still a challenging problem that limits the benefits of the lipid lowering effect. EGCG is extremely unstable in neutral and alkaline pH and degraded almost completely in a few minutes.

### SUMMARY

Embodiments of the present invention provide nanoparticle structure, including a hepatic targeted nanoparticle; and one or more substances encapsulated within the hepatic targeted nanoparticle. The nanoparticle includes chitosan (CH) and glycyrrhetinic acid (GA) covalently linked by an amide bond which occurs between an amino group of chitosan and a carboxylic acid group of GA. The one or more substances include (−)-Epigallocatechin-3-gallate (EGCG).

### BRIEF DESCRIPTION OF THE DRAWINGS

FIG. 1A depicts a chemical conjugation process for synthesizing glycyrrhetinic acid (GA) with chitosan to generate a covalently bonded chitosan-GE conjugate, in accordance with embodiments of the present invention.

FIG. 1B depicts infrared (IR) spectra of chitosan, GA, and chitosan-GA conjugate, in accordance with embodiments of the present invention.

FIG. 2 depicts nuclear magnetic resonance (NMR) spectra of chitosan, GA, and chitosan-GA conjugate, in accordance with embodiments of the present invention.

FIG. 3 depicts (−)-epigallocatechin gallate (EGCG) nano-formulation (i) particle sizes measured by dynamic light scattering (nm) and (ii) zeta potential measured by electrophoretic light scattering techniques, in accordance with embodiments of the present invention.

FIG. 4 depicts results of a kinetics study of Nano-targeted EGCG in Fetal bovine serum (FBS), Phosphate buffer Saline (PBS), Stimulated Gastric fluid (SGF), and stimulated intestinal fluid (SIF), in accordance with embodiments of the present invention.

FIG. 5A depicts plasma levels of total cholesterol of high-fat diet mice administrated orally with vehicle control (VCtrl), EGCG (30 mg/kg), void nanoparticle (void-NP), Nano EGCG (NP-EGCG) and nano-targeted EGCG (NPT-EGCG) at 30 mg/kg equivalent EGCG for 3 weeks, in accordance with embodiments of the present invention.

FIG. 5B depicts percentage reduction of plasma with total cholesterol being compared with VCtrl and void NP at week 3, in accordance with embodiments of the present invention.

FIGS. 6A and 6C depict plasma levels of free cholesterol and cholesteryl esters, respectively, of high-fat diet mice administrated orally with vehicle control (VCtrl), EGCG (30 mg/kg), void NP, Nano EGCG (NP-EGCG) and nano-targeted EGCG (NPT-EGCG) at 30 mg/kg equivalent EGCG for 3 weeks, in accordance with embodiments of the present invention.

FIGS. 6B and 6D depict percentage reduction of plasma free cholesterol, and cholesteryl esters were compared with VCtrl and void NP at week 3, in accordance with embodiments of the present invention.

FIGS. 7A-7D depict results of high-fat diet mice were administrated orally with vehicle control (VCtrl), EGCG (30 mg/kg), void NP, Nano EGCG (NP-EGCG) and nano-targeted EGCG (NPT-EGCG) at 30 mg/kg equivalent EGCG for 3 weeks, in accordance with embodiments of the present invention.

FIGS. 8A-8B depict plasma levels of HDL-C of high-fat diet mice administrated orally with vehicle control (VCtrl), EGCG (30 mg/kg), void NP, Nano EGCG (NP-EGCG) and nano-targeted EGCG (NPT-EGCG) at 30 mg/kg equivalent EGCG for 3 weeks, in accordance with embodiments of the present invention.

### DETAILED DESCRIPTION

The present invention improves the intestinal stability of EGCG by nano encapsulation using Chitosan/ascorbic acid self-assembly. Well characterized hepatic targeted EGCG nanoparticles were prepared by inventors of the present invention using glycyrrhetinic acid (GA) targeting moiety. The lipid lowering pharmacodynamic efficiency of the targeted and non-targeted EGCG nanoparticles was evaluated using nutritionally induced hypercholesterolemia mouse model. In vitro study was conducted to evaluate the effect of hepatic targeted EGCG against LDL receptor (LDL-R). Results showed that nano encapsulation of EGCG improved the stability and absorption of EGCG in the intestine by protecting the intestine from the alkaline pH and the enzymatic action of the intestine. Positively surface charged hepatic targeted nano EGCG and non-targeted nano EGCG showed a significant (***P<0.001) reduction of plasma total cholesterol, Low-density lipoprotein cholesterol (LDL-C), free cholesterol, and cholesterol esters. Further, a significant increase in plasma High-density lipoprotein cholesterol (HDL-C) was observed when compared to control after 21 days of treatment compared to the control. Western blot showed up-regulation of LDL-R 70% for hepatic targeted nano EGCG compared to the control. Hepatic targeted nano formulated EGCG seems to be a natural therapeutic agent that is therapeutically effective against hypercholesterolemia.

Green tea (Camellia sinensis) is a highly consumed beverage worldwide. The habitual consumption of green tea has been associated with health benefits including chemo-preventive biological. The biological activities of green tea are mediated by green tea's polyphenolic active ingredients known as catechins, which include -epigallocatechin gallate, -epigallocatechin (EGC), (−)-epicatechin gallate (ECG), and (−)-epicatechin. EGCG is the major catechin in green tea and accounts for 50-80% representing 200-300 mg/brewed cup of green tea (C JURADO-CORONEL, J., ECHEVERRIA, V., HIDALGO, O. A., GONZALEZ, J., ALIEV, G. & E BARRETO, G. 2016, Implication of green tea as a possible therapeutic approach for Parkinson disease, CNS & Neurological Disorders-Drug Targets (Formerly Current Drug Targets-CNS & Neurological Disorders), 15, 292-300; RANA, A., RANA, S. & KUMAR, S. 2021, Phytotherapy with active tea constituents: a review, Environmental Chemistry Letters, 19(22) DOI:10.1007/s10311-020-01154-y). Research is mostly dealing with EGCG as chemo preventive agent for several diseases including different types of cancer, Alzheimer's disease, hypertension, obesity and weight control, and cardiovascular disease. The role of EGCG in multi-diseases management can be attributed to the antioxidant and anti-inflammatory properties of catechins' active ingredients.

Evidence from experimental studies indicates that EGCG retards the development or progression of atherosclerosis in apolipoprotein E-deficient mice (CHYU, K.-Y., BABBIDGE, S. M., ZHAO, X., DANDILLAYA, R., RIETVELD, A. G., YANO, J., DIMAYUGA, P., CERCEK, B. & SHAH, P. K. 2004, Differential effects of green tea-derived catechin on developing versus established atherosclerosis in apolipoprotein E-null mice, Circulation, 109, 2448-2453 and hypercholesterolemic hamsters (Vinson, J. A., TEUFEL, K. & WU, N. 2004. et al., 2004. Green and black teas inhibit atherosclerosis by lipid, antioxidant, and fibrinolytic mechanisms, Journal of agricultural and food chemistry, 52, 3661-3665.). Studies have shown that ECGC antioxidant activities effectively inhibit low density lipoprotein (LDL) oxidation and lipid peroxidation (SKRZYDLEWSKA, E., OSTROWSKA, J., FARBISZEWSKI, R. & MICHALAK, K. 2002. Protective effect of green tea against lipid peroxidation in the rat liver, blood serum and the brain. Phytomedicine, 9, 232-238).

ECGC can reduce lipid and the cardiovascular disease by two mechanisms, in the intestine and liver. In the intestine, EGCG effectively lowers the intestinal absorption of ingested lipids. The potent inhibitory effect appears to be associated with formation of complexes with lipids and lipolytic enzymes, thereby interfering with the luminal processes of emulsification, hydrolysis, micellar solubilization, and subsequent uptake of lipids such as cholesterol and α-tocopherol, with little or a moderate effect on less hydrophobic lipids such as retinol and fatty acid. In hepatocytes, interesting results were recorded by Cui et al. that ECGC which identifies suppression of PCSK9 production by promoting nuclear FoxO3a, and reducing nuclear HNF1α, resulting in up-regulated LDLR expression and LDL uptake in hepatocytes, thereby inhibits liver and circulating PCSK9 levels, and ultimately lowers LDL-C levels (Cui et al., 2020). In addition, it was demonstrated that EGCG potently inhibits the invitro activity of Hydroxy-3-methyl-glutaryl-CoA reductase (HMGR) which is the rate-controlling enzyme of cholesterol synthesis by competitively binding to the cofactor site of the reductase (CUCCIOLONI, M., MOZZICAFREDDO, M., SPINA, M., TRAN, C. N., FALCONI, M., ELEUTERI, A. M. & ANGELETTI, M. 2011, Epigallocatechin-3-gallate potently inhibits the in vitro activity of hydroxy-3-methyl-glutaryl-CoA reductase. Journal of lipid research, 52, 897-907).

The stability of EGCG is still a challenging problem limiting the EGCG benefit of the lipid lowering effect where EGCG is extremely unstable and degraded almost completely in a few minutes at neutral and alkaline solutions, whereas in acidic solutions (pH<4) EGCG is very stable. Thus, it is possible that the partial intestinal absorption may be due to instability of EGCG in the intestine where the pH is neutral or alkaline.

The present invention solves the instability problem of green tea EGCG by nanoencapsulation inside chitosan nanoparticles, which improves the stability and absorption in the intestine by protecting the intestine from the alkaline pH and the enzymatic action of the intestine. In addition, hepatic targeted chitosan-glycyrrhetinic acid EGCG nanoparticles were synthesized and evaluated for targeting the PSCK-9 inhibitory pathway.

The lipotropic effect of the nano encapsulated EGCG was evaluated in vivo by a pharmacodynamic study using a nutritionally induced hypercholesterolemia mouse model. In vitro study was conducted to evaluate the effect of hepatic targeted nano EGCG to up regulate the LDL-receptors in the hepatocytes.

### Materials and Methods

### Chemicals

(−)-epigallocatechin gallate (EGCG) (95% purity), L-Ascorbic acid, Low molecular weight Chitosan (50-150 kD), Sodium triphosphate pentabasic, Ethanol, and Amicon ultra-0.5 centrifugal filters, were purchased from Sigma-Aldrich.

### (−)-Epigallocatechin Gallate and Targeted (−)-Epigallocatechin Gallate Nanoparticles

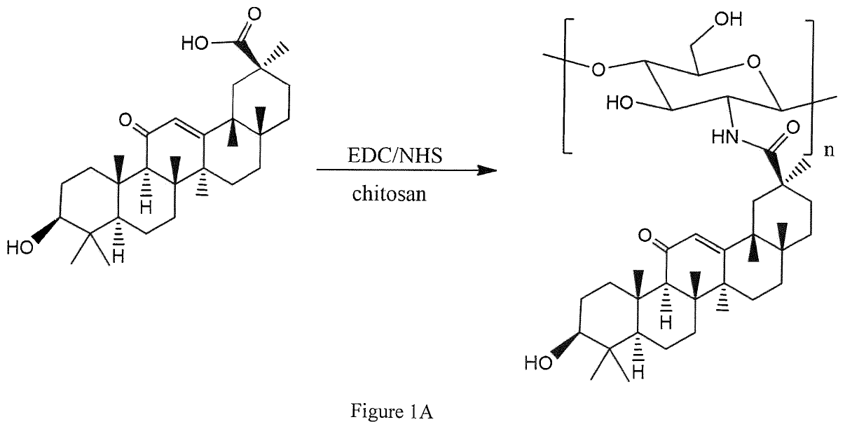

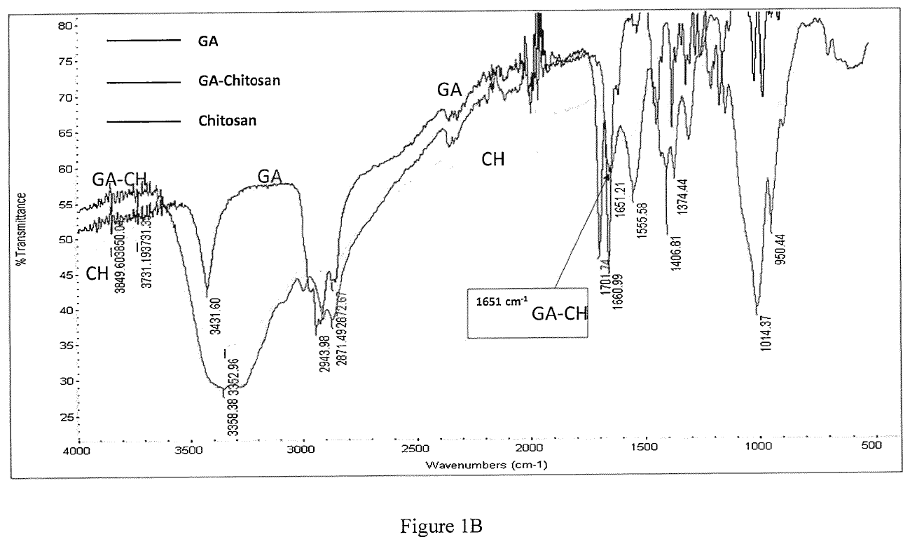

FIG. 1A depicts a chemical conjugation process for synthesizing glycyrrhetinic acid (GA) with chitosan to generate a covalently bonded chitosan-GE conjugate, in accordance with embodiments of the present invention. A self-assembly encapsulation technique was utilized for preparation of (−)-epigallocatechin gallate (EGCG) chitosan encapsulated nanoparticles. Autonomous interaction between Chitosan (encapsulating agent) and EGCG (bioactive) was enhanced by tripolyphosphate (TPP). EGCG nanoparticles were prepared by the drop-wise addition of TPP solution (0.1%, w/v) to a doubled filtered (45 μm filter, Millipore, USA) chitosan solution (0.1%, w/v, in 0.08%, w/v, ascorbic acid) containing EGCG (0.05%, w/v) at ambient conditions and under magnetic stirring (700 rpm) for 30 min. The mass ratio of CS:TPP in the final mixture was 3:1. Nanoparticles (NPs) were collected by centrifugation at 40 000×g for 45 min and freeze-dried at −80° C. for 24 h before use. Void nanoparticles were formed of non-loaded chitosan nanoparticles (free of EGCG) following the same preparation protocol without addition of EGCG. For hepatic targeted EGCG encapsulated nanoparticles, chitosan-glycyrrhetinic acid was used instead of chitosan following the same procedures and ratios. Chemical conjugation of chitosan and glycyrrhetinic acid was done as follows. 126 mg 1-ethyl-3-(3-dimethylaminopropyl) carbodiimide hydrochloride (EDC HCl) and 63 mg N-hydroxy succinimide were added to 160 mg GA solution in DMF. The solution was mixed with 2% chitosan in acetic acid and stirred at room temperature. After 48 hours (h), the mixture was precipitated with acetone, and the precipitate was washed with 60% ethanol and ether. The final product was obtained after vacuum drying and characterized by FT-IR and 1-HNMR techniques.

FIG. 1B depicts infrared (IR) spectra of chitosan (CH), glycyrrhetinic acid (GA), and GA-Chitosan (GA-CH) conjugate, in accordance with embodiments of the present invention.

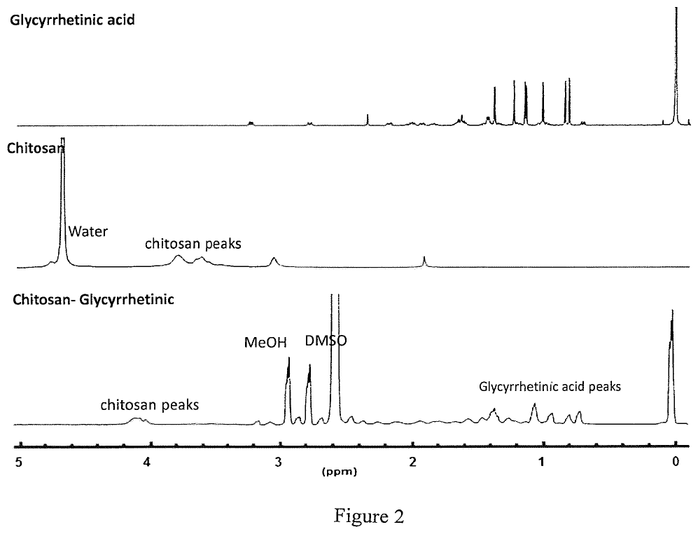

FIG. 2 depicts nuclear magnetic resonance (NMR) spectra of chitosan, GA, and GA-CH conjugate, in accordance with embodiments of the present invention.

### Encapsulation Efficiency and Loading Ratio

The encapsulation efficiency of (−)-epigallocatechin gallate (EGCG) was determined by analyzing the EGCG amount encapsulated in the nanoparticles compared to the amount of EGCG added initially. After lyophilization, a weighed nano formulated powder was disintegrated by acetic acid and the liberated EGCG was quantified with ultraviolet-visible (UV-vis) spectrophotometer against an established calibration curve (Nanodrop 2000C ThermoFisher Scientific, Waltham, MA, USA). The EGCG encapsulation efficiency was calculated according to equation (1). The EGCG loading ratio was determined by measuring the amount of EGCG in the nanoparticles and the weight of whole nanoparticles according to equation (2).

$$Encapsulation efficiency = \frac{EGCG amount encapsulated}{EGCG amount added initially} \times 100 \qquad ( 1 )$$

$$Loading ratio = \frac{EGCG amount encapsulated}{Total weight of nanoparticles} \times 100 \qquad ( 2 )$$

### In Vitro Release Kinetics

The release kinetics of (−)-epigallocatechin gallate (EGCG) from each nano formulation was studied in different media, namely Fetal Bovine Serum (FBS), Phosphate Buffered Saline (PBS), Stimulated Gastric Fluid (SGF) and Stimulated Intestinal Fluid (SIF). For this cumulative release kinetic study, known amounts of each EGCG nano formulation were suspended in 5 ml of each medium and incubated at 37° C. At predetermined time intervals (0.5, 1.0, 3.0, 6, 12, and 24 h), 500 μl of the solution was filtered through Millipore tubes containing a kD membrane to separate released EGCG from the nanoparticles, and the amount of EGCG released was measured using a UV-VIS spectrophotometer against an EGCG calibration curve.

### Animals

High fat diet male C57BL/6 mice aged 4-5 weeks and weighing 20-25 g were purchased from Taconic Biosciences, Inc. (Germantown, NY, USA). All animal studies were conducted at the animal facility of the Veteran Affairs Medical Center (VAMC) (Albany, NY, USA) in accordance with institutional guidelines for humane animal treatment and approved by the Institutional Animal Care & Use Committee (IACUC) at VAMC. Animals were maintained under specific pathogen-free conditions and housed under controlled conditions of temperature (20-24° C.) and humidity (60-70%) and 12 h light/dark cycle with ad libitum access to water and food. The mice were grouped into five arms (n=5). Administrated orally to the mice were water for vehicle control (VCtrl), Void nanoparticle (void NP), EGCG (30 mg/kg), Nano EGCG (NP-EGCG) and Nano-targeted EGCG (NPT-EGCG) at 30 mg/kg equivalent EGCG for 3 weeks. Blood was collected from retro-orbital after the 24 h, week 1, week 2 and week 3 treatment to measure the levels of plasma total cholesterol, LDL-C, HDL, free and cholesteryl esters. Animals were humanely sacrificed, and liver tissue was collected.

### Plasma Lipid Profile

The plasma contents of total cholesterol, LDL-C, HDL-C, free and cholesteryl esters were measured with the Cholesterol-HDL and LDL/VLDL assay kit according to the manufacturer's instructions (Abcam, Boston, MA, USA).

### Effect on LDL-R by Western Blots Assay in Liver Tissues

To study the expression of LDL-R, samples of liver tissues were homogenized in lysis buffer (50 mmol/l Tris, pH7.5, 150 mmol/l sodium chloride, 1 mmol/l EDTA, 0.5% NP40, 0.1% SDS, protease inhibitor cocktail (Roche, Mannheim). Proteins (50 μg) from each sample were loaded on 12% SDS-polyacrylamide gel, and separated proteins from each sample were transferred to Immobilon-PSQ PVDF membranes (Millipore, Billerica, MA, USA) with the Mini Trans-Blot Cell (Bio-Rad Laboratories). The membrane in each sample was blocked with a solution of 2% BSA in Tris-buffered saline and incubated with primary antibodies to LDL-R (Invitrogen, Rockford, IL, USA) at 4° C. overnight and washed. The proteins were detected with HRP-conjugated secondary antibodies (Cell Signaling Technology, Danvers, MA) for 1 h at room temperature. Detection was done using enhanced chemiluminescence (Thermo Scientific, Grand Island, NY). GAPDH was used as the control (Santa Cruz Biotechnology, Inc, Dallas, Texas). Membranes were scanned and intensities of the bands were analyzed using ImageJ analyzer software (National Institutes of Health, Bethesda, MD, USA).

### Statistical Analysis

Statistical analysis was performed using GraphPad Prism (San Diego, CA, USA) (Wilkinson, 1956). All data are presented as mean±standard deviation of the mean. The Mann-Whitney nonparametric test was used to determine differences in the groups. ***P<0.001, **P<0.01, *P<0.05 were considered as significant.

### Results

### Characterization of Encapsulated EGCG and Targeted EGCG Nanoparticles

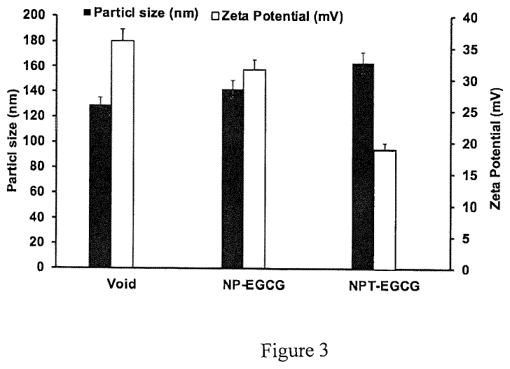

FIG. 3 depicts (−)-epigallocatechin gallate (EGCG) nano-formulation (i) particle sizes measured by dynamic light scattering (nm) and (ii) zeta potential measured by electrophoretic light scattering techniques, in accordance with embodiments of the present invention. Self-assembly nanoencapsulation technique was applied to encapsulate (−)-epigallocatechin gallate (EGCG) using chitosan tripolyphosphate encapsulation system.

EGCG, Targeted EGCG and void nanoparticles were successfully prepared.

The EGCG nanoparticles were prepared by dissolving 100 mg EGCG in 94 ml distilled water (DW) which is next mixed with 5 ml 1% chitosan solution followed by stirring for 30 minutes to form a chitosan-EGCG mixture. Then, 1 ml tripolyphosphate (TPP) is added to the chitosan-EGCG mixture, followed by vigorous stirring for 2 hours.

The void nanoparticles were prepared by diluting a 5 ml 1% chitosan solution to 99 ml by addition of 94 ml DW and then stirring for 30 minutes, after which 1 ml TPP (10 mg/ml) is added, followed by vigorous stirring for 2 hours.

Particle size was measured by dynamic light scattering (DLS) and zeta potential was measured by electrophoretic light scattering (ELS) techniques.

The particle sizes of the EGCG, Targeted EGCG and void nanoparticles were 135.6, 156.3 nm, and 123.1 nm and their corresponding zeta potentials were 31.6 mV, 18.9 mV, and 36.1 mV, respectively.

FIG. 3 depicts one standard deviation ranges of particle size and zeta potential for EGCG, Targeted EGCG and void nanoparticles.

From FIG. 3, the one standard deviation range of particles size of the EGCG, Targeted EGCG, and void nanoparticles are 127-144 nm, 148-165 nm, and 116-130 nm, respectively, and the zeta potential of the EGCG, Targeted EGCG, and void nanoparticles are 30-33 mV, 17-20 mV, and 34-38 mV, respectively.

From FIG. 3, the two standard deviation range of particles size of the EGCG, Targeted EGCG, and void nanoparticles are 119-152 nm, 140-173 nm, and 110-137 nm, respectively, and the zeta potential of the EGCG, Targeted EGCG, and void nanoparticles are 28-35 mV, 16-22 mV, and 32-40 mV, respectively.

EGCG encapsulation efficiencies were 98% and 95.2%, while EGCG loading ratios were 12.4% and 11.3% for EGCG targeted nanoparticles and EGCG nanoparticles, respectively, which reflect the success of achieving excellent encapsulation efficiency and loading percentage that are in accordance with previously published work.

### In Vitro Release Kinetics

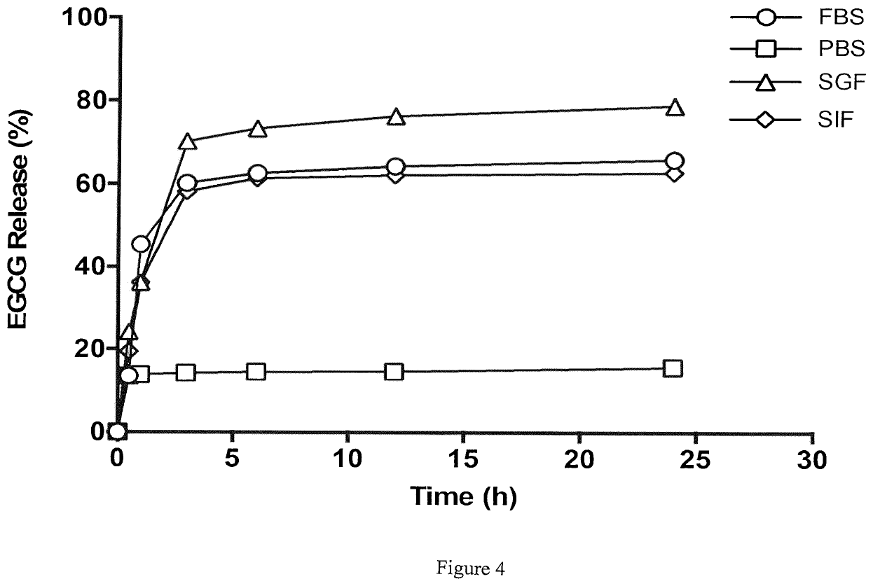

FIG. 4 depicts results of a kinetics study of Nano-targeted EGCG in Fetal bovine serum (FBS), Phosphate buffer Saline (PBS), Stimulated Gastric fluid (SGF), and stimulated intestinal fluid (SIF), in accordance with embodiments of the present invention. The release kinetic profiles of EGCG NPs and Targeted Nano NPs were conducted in Stimulated Gastric Fluid (SGF), Stimulated Intestinal Fluid (SIF), Fetal Bovine Serum (FBS) and Phosphate Buffer Saline (PBS). Release of EGCG shows similar trend of release in the corresponding medium. The maximum release was observed in the stimulated gastric fluid about 90% over 24 h. The minimum release was recorded in PBS about 10% for 24 h reflecting high stability of the prepared nanoparticles. The release kinetics in FBS & SIF media were close to 70% for 24 h.

### Plasma Lipid Profile

Change in levels of total plasma cholesterol of high-fat diet mice administrated orally with vehicle control (VCtrl), EGCG (30 mg/kg), void NP, Nano EGCG (NP-EGCG) and nano-targeted EGCG (NPT-EGCG) at 30 mg/kg equivalent EGCG for 3 weeks was determined from blood collected for 24 h, week 1, week 2 and week 3.

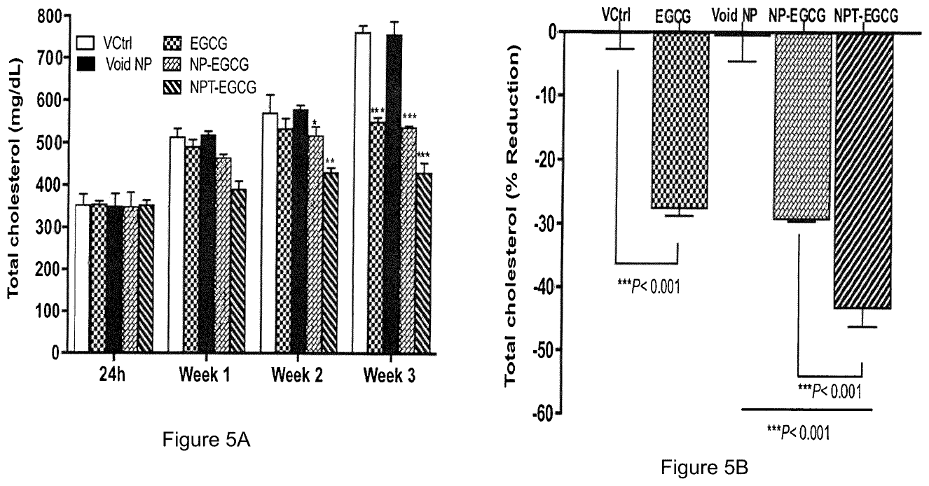

FIG. 5A depicts plasma levels of total cholesterol of high-fat diet mice administrated orally with vehicle control (VCtrl), EGCG (30 mg/kg), void NP, Nano EGCG (NP-EGCG) and nano-targeted EGCG (NPT-EGCG) at 30 mg/kg equivalent EGCG for 3 weeks, in accordance with embodiments of the present invention. Blood was collected at 24 h, week 1, week 2 and week 3. A significant decrease in total plasma cholesterol levels was observed with NP-EGCG and NPT-EGCG at week 2 and week 3 compared to void NP.

FIG. 5B depicts percentage reduction of plasma with total cholesterol being compared with VCtrl and void NP at week 3, in accordance with embodiments of the present invention. Values are presented as mean±S.D. Statistical analysis were conducted using Mann-Whitney nonparametric test, ***P<0.001, **P<0.01, *P<0.05 versus respective controls. Nano-targeted EGCG was statistically significant different versus Nano-EGCG, ***P<0.001. At week 3, EGCG, NP-EGCG and NPT-EGCG showed 28%, 30%, 44% decrease (***P<0.001), respectively, compared to vehicle control and void NP.

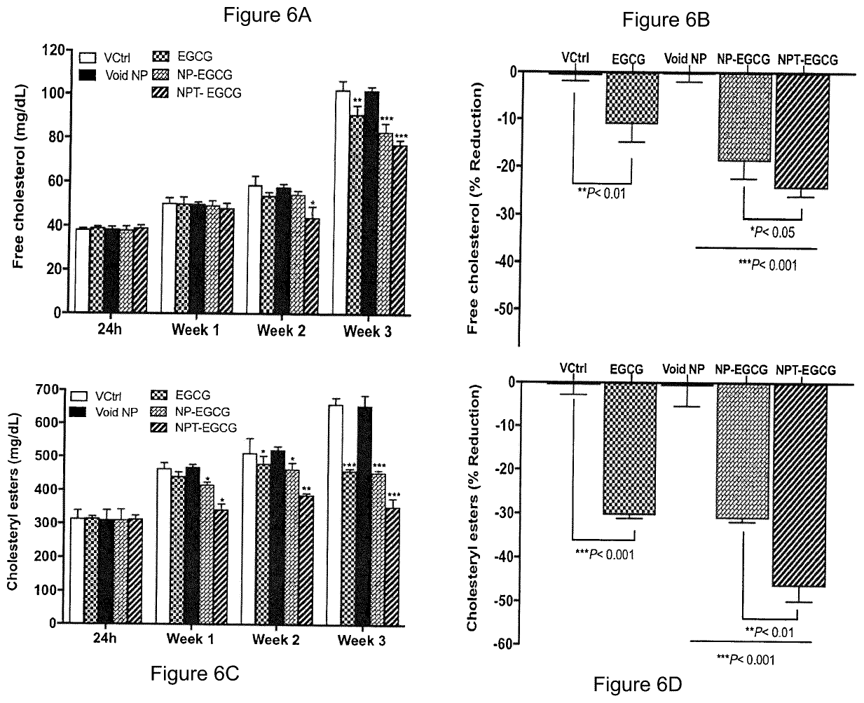

FIGS. 6A and 6C depict plasma levels of free cholesterol and cholesteryl esters, respectively, of high-fat diet mice administrated orally with vehicle control (VCtrl), EGCG (30 mg/kg), void nanoparticle (void-NP), Nano EGCG (NP-EGCG) and nano-targeted EGCG (NPT-EGCG) at 30 mg/kg equivalent EGCG for 3 weeks, in accordance with embodiments of the present invention.

FIGS. 6B and 6D depict percentage reduction of plasma free cholesterol, and cholesteryl esters were compared with VCtrl and void NP at week 3, in accordance with embodiments of the present invention.

In FIGS. 6A-6D, blood was collected at 24 h, week 1, week 2 and week 3. Values are presented as mean±S.D. Statistical analysis were conducted using Mann-Whitney nonparametric test, ***P<0.001, **P<0.01, *P<0.05.

A significant decrease (***P<0.001, **P<0.01) in levels of free cholesterol in plasma of high fat diet mice was observed with EGCG, NP-EGCG and NPT-EGCG week 3 compared to vehicle control and void NP (FIG. 6A). Further, a significant decrease (***P<0.001) in cholesteryl esters was observed with EGCG, NP-EGCG and NPT-EGCG week 2 and week 3 (FIG. 6C). At week 3, free cholesterol and cholesteryl esters showed 11%, 19%, 25% and 30%, 32% and 46% reduction compared to vehicle control and void NP (FIGS. 6B and 6D).

A significant decrease (**P<0.01) in plasma LDL-C levels of high fat diet mice were observed with NP-EGCG and NPT-EGCG week 2 and week 3 compared to vehicle control and void NP respectively (FIG. 6A). As depicted in FIG. 6B, 3%, 10% and 12% decrease in plasma LDL-C were observed with EGCG, NP-EGCG and NPT-EGCG, respectively, in week 3. Further, liver LDL-R expression increased 4, 5.5 and 9-fold with EGCG, NP-EGCG and NPT-EGCG, respectively, compared to vehicle control and void NP (FIG. 6C).

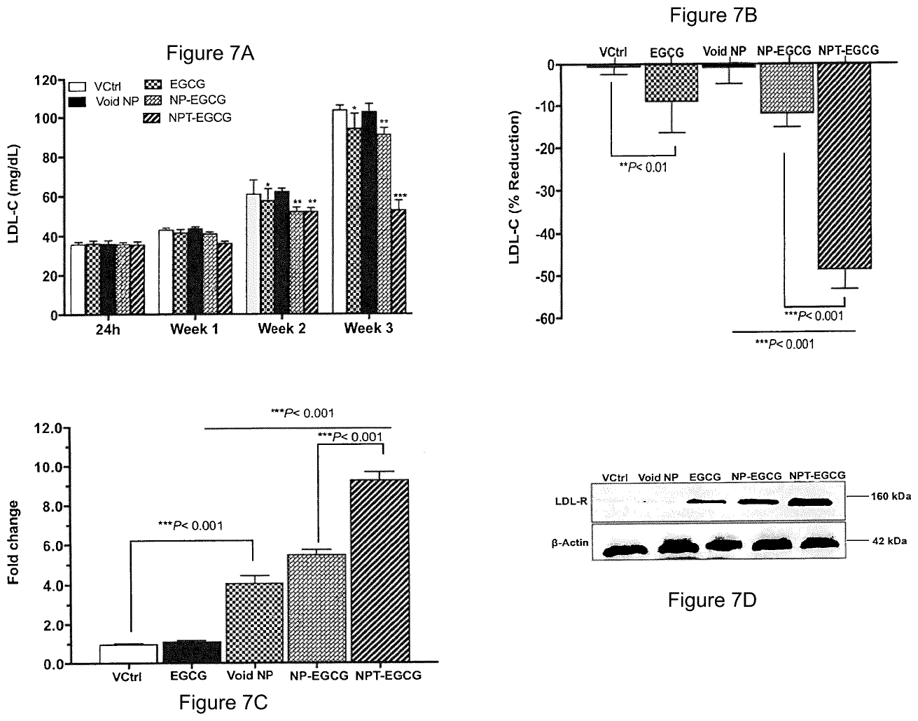

FIGS. 7A-7D depict results of high-fat diet mice were administrated orally with vehicle control (VCtrl), EGCG (30 mg/kg), void NP, Nano EGCG (NP-EGCG) and nano-targeted EGCG (NPT-EGCG) at 30 mg/kg equivalent EGCG for 3 weeks, in accordance with embodiments of the present invention. Blood was collected at 24 h, week 1, week 2 and week 3.

FIGS. 7A-7B depict plasma levels of LDL-C of the high-fat diet mice, FIG. 7C depicts fold change, and FIG. 7D depicts a representative image of a western blot showing LDL-R expression.

Values in FIGS. 7A-7C are presented as mean±S.D. Statistical analysis were conducted using Mann-Whitney nonparametric test, ***P<0.001, **P<0.01, *P<0.05.

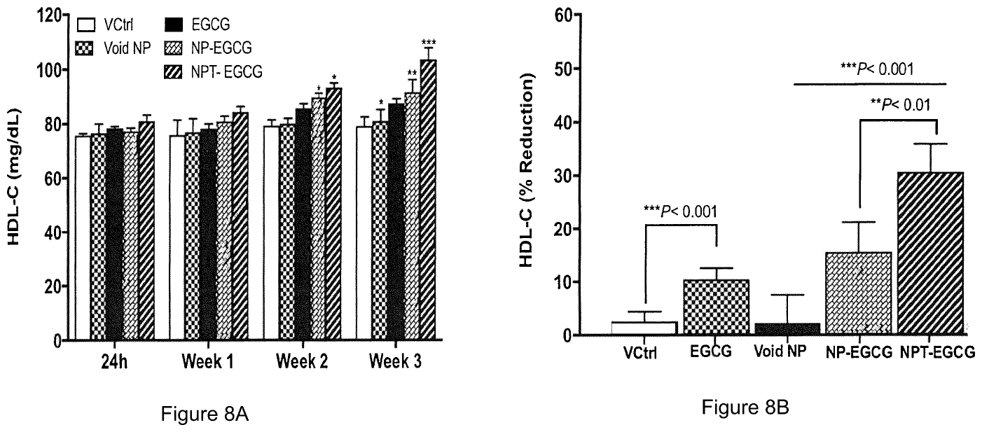

FIGS. 8A-8B depict plasma levels of HDL-C of high-fat diet mice administrated orally with vehicle control (VCtrl), EGCG (30 mg/kg), void NP, Nano EGCG (NP-EGCG) and nano-targeted EGCG (NPT-EGCG) at 30 mg/kg equivalent EGCG for 3 weeks, in accordance with embodiments of the present invention. Blood was collected at 24 h, week 1, week 2 and week 3. B and D) Percentage reduction of plasma HDL-C was compared with VCtrl and void NP at week 3. Values are presented as mean±S.D. Statistical analysis were conducted using Mann-Whitney nonparametric test, ***P<0.001, **P<0.01, *P<0.05.

FIG. 8 shows that the levels of plasma HDL-C levels of high fat diet mice were significantly elevated with NP-EGCG and NPT-EGCG week 2 and week 3 compared to vehicle control (FIG. 8A). Further, at week 3, 10%, 16% and 31% increase in plasma HDL-C were observed with EGCG, NP-EGCG and NPT-EGCG, respectively (FIG. 8B).

### DISCUSSION

The preceding test results demonstrate that the nanoparticle structure provided by embodiments enables EGCG to have sufficient stability to provide a remarkable lipid lowering effect and hence ameliorative effect cardiovascular system. Such lipid lowering action of EGCG may be attributed to inhibition of the intestinal absorption of ingested lipids and to inhibition of PCSK-9 activity and up-regulation of the LDL-R in liver tissues.

EGCG is unstable at intestinal pH and not readily absorbed, with small percentages of orally ingested catechins appearing in the blood in rats and humans. The present invention improves the stability of EGCG by nano encapsulation into self-assembled chitosan-ascorbic acid. On the other hand, the up-regulation effect on LDL-R in livers was improved by synthesis of hepatic targeted EGCG using glycyrrhetinic acid targeting moiety through formation of chitosan-glycyrrhetinic acid conjugate. The in vitro release kinetics study showed slow release of EGCG in the stimulated intestinal fluids reflecting better stability of the nanoformulation against the intestinal pH and digestive enzymes compared to the free EGCG.

Results of the Pharmacodynamic study are highly promising, due to significant reduction of plasma total cholesterol, LDL-C, free cholesterol and cholesterol esters and increase of HDL-C. The nano encapsulated EGCG (hepatic targeted/non-targeted) showed significant (***P<reduction of LDL-C compared to free EGCG. Moreover, hepatic targeted nano EGCG reduced the plasma levels of LDL-C compared to the nontargeted nano green tea. The LDL-C reduction efficiency of hepatic targeted nano EGCG in this study is highest compared to previously recorded. This LDL-C reduction efficiency might be attributed to the higher stability of the present inventive nano formulations in the gut protecting the EGCG from the intestinal enzymes and alkaline pH demonstrating prolonged inhibition of intestinal lipid absorption. The hepatic targeted nano EGCG showed about 100% LDL-C reduction efficiency compared to non-targeted nano green tea that might be attributed to the successful delivery of EGCG to the hepatocytes at higher concentration promoting higher up-regulation of LDL-R in the liver. Results of Western blot experiment confirmed the assumed mechanism of the present invention, where significant up-regulation of LDL-R (**P<0.01 and ***P<0.001) to approximately 8 times targeted nano EGCG group compared to 4 times for non-targeted nano EGCG.

### Nanoparticle Structure

The inventive nanoparticle structure includes a positively surface charged hepatic targeted nanoparticle and one or more substances encapsulated within the nanoparticle. The hepatic targeted nanoparticle includes chitosan and glycyrrhetinic acid (GA) covalently linked by an amide bond which occurs between an amino group of chitosan and a carboxylic acid group of GA. The GA functions as a hepatic targeting moiety. The substances encapsulated within the nanoparticle include (i) EGCG (ii) EGCG and ascorbic acid; (iii) EGCG and other polyphenols; (iv) EGCG and ascorbic acid and other polyphenols. The other polyphenols in (iii) and (iv) include one or more polyphenols selected from the group consisting of resveratrol, curcumin, flavonoids, isoflavones, and combinations thereof.

The nanoparticle structure may be administered to a mammal to lower lipids in the mammal. In one embodiment, the mammal is a human being. In one embodiment the mammal is a non-human mammal.

While embodiments of the present invention have been described herein for purposes of illustration, many modifications and changes will become apparent to those skilled in the art. Accordingly, the appended claims are intended to encompass all such modifications and changes as fall within the true spirit and scope of this invention.

What is claimed is:

1. A nanoparticle structure, comprising:
a positively surface charged hepatic targeted nanoparticle; and
one or more substances nano-encapsulated within the hepatic targeted nanoparticle,
wherein the targeted nanoparticle comprises chitosan and glycyrrhetinic acid (GA) covalently linked by an amide bond which occurs between an amino group of chitosan and a carboxylic acid group of GA, and
wherein the one or more substances comprise (−)-Epigallocatechin-3-gallate (EGCG).

2. The nanoparticle structure of claim 1, further comprising one or more substances further comprise one or more polyphenols selected from the group consisting of resveratrol, curcumin, flavonoids, isoflavones, and combinations thereof.

3. The nanoparticle structure of claim 2, further comprising ascorbic acid.

4. The nanoparticle structure of claim 1, further comprising ascorbic acid.

5. The nanoparticle structure of claim 1, wherein the targeted nanoparticle has a particle size in a range of 140 to 173 nm.

6. The nanoparticle structure of claim 1, wherein the targeted nanoparticle has a zeta potential size in a range of 16 to 22 mV.

7. The nanoparticle structure of claim 1, wherein the nanoparticle has an EGCG encapsulation efficiencies of about 98%.

8. The nanoparticle structure of claim 1, wherein the nanoparticle has an EGCG loading ratio of about 12%.

9. The nanoparticle structure of claim 1, wherein the nano-encapsulation of the EGCG is configured to improve a stability of the EGCG and to improve an absorption of EGCG in an intestine of a mammal by protecting the intestine from the alkaline pH and the enzymatic action of the intestine.

10. A method for lowering lipids in a mammal, said method comprising:
administering the nanoparticle structure of claim 1 to the mammal.

11. The method of claim 10, wherein the mammal is a human being.

12. The method of claim 10, wherein the mammal is a non-human mammal.

13. The method of claim 10, wherein after said administering the nanoparticle structure, the nanoparticle structure enters an intestine of the mammal and the nano-encapsulation of the EGCG improves an absorption of the EGCG in the intestine by protecting the intestine from the alkaline pH and the enzymatic action of the intestine.

14. The method of claim 10, wherein after 21 days following said administering the nanoparticle structure to the mammal, plasma total cholesterol, low-density lipoprotein cholesterol (LDL-C), free cholesterol, and cholesterol esters are reduced in the mammal and plasma high-density lipoprotein cholesterol (HDL-C) is increased in the mammal in comparison with a control.
